(19) United States
(12) Patent Application Publication
(10) Pub. No.: US 2025/0378925 A1
(43) Pub. Date: Dec. 11, 2025

(54) DIGITAL IMAGING AND ARTIFICIAL INTELLIGENCE (AI)-BASED SYSTEMS AND METHODS FOR ANALYZING PRODUCT IMAGES

(71) Applicant: The Procter & Gamble Company, Cincinnati, OH (US)

(72) Inventors: Sol Melissa ESCOBAR, Mason, OH (US); Kelly L. ANDERSON, Mason, OH (US)

(21) Appl. No.: 19/228,823

(22) Filed: Jun. 5, 2025

Related U.S. Application Data
(60) Provisional application No. 63/656,757, filed on Jun. 6, 2024.

(51) Int. Cl. G16H 20/00; G06Q 30/0601; G06V 10/764; G16H 50/30

(52) U.S. Cl. CPC G16H 20/00; G06Q 30/0631; G06V 10/764; G16H 50/30

(57) ABSTRACT

Digital imaging and artificial intelligence (AI)-based systems and methods are described for analyzing product images and making product recommendations. An imaging application (app) receives a set of digital image(s) comprising pixel data depicting a product. A product-based learning model is applied to the pixel data in order to predict one or more product identifiers corresponding to one or more products depicted within pixel data of a plurality of training images. One or more risk factors associated with the user are predicted based on applying a risk factor model to personal parameters of the user and the product identifier. One or more products and/or one or more routines are recommended, and output, for the user, based on applying a recommender 10 model to the product identifier, the personal parameters, the risk factors, one or more goals associated with the user, and, optionally, one or more preferences associated with the user.

### TECHNICAL FIELD

The present disclosure generally relates to digital imaging and artificial intelligence (AI)-based systems and methods, and more particularly to digital imaging and AI-based systems and methods configured to analyze images of products and parameters associated with users and make product and/or routine recommendations.

### BACKGROUND

A wide range of oral care products, including toothpastes, mouthwashes, toothbrushes, floss, whitening treatments, etc., are designed to maintain oral hygiene and prevent conditions such as gingivitis, periodontitis, and tooth decay. However, the vast array of available products can often lead to confusion among users. Many individuals are unsure about which products are the right fit for their specific oral care concerns and/or oral care goals. This uncertainty is further compounded by a lack of knowledge about oral care conditions and related symptoms, as well as the specific benefits and attributes of different oral care products. Moreover, maintaining consistent oral care routines to address oral care concerns and/or oral care goals is a challenge for many individuals, due to a variety of factors, including lack of time, forgetfulness, and lack of awareness about best practices in oral care.

For the foregoing reasons, there is a need for systems and methods for improving the oral health experience of consumers.

### SUMMARY

Generally, as described herein, digital imaging and artificial intelligence (AI)-based systems and methods are described for analyzing images of products and parameters associated with users and making product and/or routine recommendations. Such digital imaging and AI-based systems and methods provide a technical solution for overcoming problems that arise from the difficulties in identifying and using various products that are ideally suited to the specific oral care conditions and preferences of particular individuals in a clinically effective manner.

The digital imaging and AI-based systems and methods as described herein allow a user to submit one or more images to imaging server(s) (e.g., including its one or more processors), or otherwise a computing device (e.g., such as locally on the user's mobile device), where the imaging server(s) or user computing device implements or executes an AI-based learning model trained with pixel data of potentially 10,000 s (or more) images depicting products. The artificial intelligence model (e.g., a product-based learning model) may generate, based on pixel data of a given image, one or more product identifiers corresponding to the one or more products depicted within the pixel data of the image. For example, an image of a product can comprise pixels or pixel data indicative of specific product features from the images, such as the product category, brand, variant, form, and packaging. The generated product identifier(s) are then used to identify the exact product and link it to additional data sources to gather more detailed information about the product, such as formulation specifications, product traits, packaging traits, and clinical indications. In addition to the image-based product analysis, the digital imaging and AI-based systems and methods also obtain user input including personal parameters, goals, and preferences. This information, along with the product data, is used to predict risk factors associated with the user. The digital imaging and AI-based systems and methods then recommend specific oral care products and/or routines for the user, based on these predicted risk factors and the user's goals and preferences. The system's output includes a feedback indication that provides the user with the recommended products and/or routines. In some embodiments, the feedback may be transmitted via a computer network to a user computing device of the user for rendering on a display screen. In other embodiments, no transmission to the imaging server of the user's specific image occurs, where the feedback indication may instead be generated by the artificial intelligence model executing and/or implemented locally on the user's mobile device and rendered, by a processor of the mobile device, on a display screen of the mobile device. In various embodiments, the feedback indication can take various forms including, for example, a qualitative rating, a numeric assessment, a visual projection, informational text, or a categorical rating.

The digital imaging and AI-based systems and methods as described herein provide a technical solution to the problem of oral care product selection. By leveraging AI and digital imaging, the digital imaging and artificial intelligence-based systems and methods are able to provide personalized product and/or routine recommendations that are tailored to the specific oral care products available to the user, as well as the specific oral care conditions and preferences of the user, which not only simplifies the product selection process for the user but also enhances the effectiveness of their oral care routine and encourages adherence to recommended oral care practices, enhancing the individual's overall oral care experience.

The disclosed digital imaging and artificial intelligence-based systems and methods can provide various features or benefits over the existing art, including, for example, the ability to instantaneously (e.g., in real-time or near real-time) detect and identify one or more products, implements, or appliances from a digital library of identifiable products, implements, or appliances by analyzing images or videos captured in various contexts, such as on a bathroom counter amidst non-oral care items. Additionally, the digital imaging and artificial intelligence-based systems and methods can extract and relay data and information about the interactions or relationships among the products, implements, or appliances, including details on product formulation, specifications, and attributes, and their correlation to the treatment of various oral care conditions (e.g., including oral care conditions of a particular user).

Specifically, in the oral care context, the disclosed digital imaging and artificial intelligence-based systems and methods offer distinct benefits. The digital imaging and artificial intelligence-based systems and methods can alleviate confusion caused by the wide range of available products. By delivering personalized recommendations that consider individual parameters and oral care requirements, the digital imaging and artificial intelligence-based systems and methods assist users in selecting the appropriate oral care products for their specific conditions, and support the maintenance of recommended oral care habits, such as brushing twice daily with fluoride toothpaste for two minutes and regular flossing, leading to better oral hygiene and overall health.

In some aspects, the techniques described herein relate to a digital imaging and artificial intelligence (AI)-based system configured to analyze images of products and parameters associated with users and make product and/or routine recommendations. The system includes one or more processors, an imaging application (app) comprising computing instructions configured to execute on the one or more processors, a product-based learning model, a risk factor model, and a recommender model. The product-based learning model is accessible by the imaging app and trained with pixel data of a plurality of training images depicting one or more products. The product-based learning model is trained to output product predictions of one or more product identifiers corresponding to the one or more products depicted within the pixel data of the plurality of training images. The risk factor model, which may be a risk factor learning model, is also accessible by the imaging app and trained with personal parameters associated with each of a plurality of individuals, and one or more products used by each of the plurality of individuals. The risk factor model is trained to output one or more predicted risk factors associated with each of the plurality of individuals based on the personal parameters associated with each of the plurality of individuals, and the one or more products used by each of the plurality of individuals. The recommender model, which may be a recommender learning model, is accessible by the imaging app and trained with the personal parameters associated with each of the plurality of individuals, the one or more products used by each of the plurality of individuals, the one or more predicted risk factors associated with each of the plurality of individuals, one or more goals associated with each of the plurality of individuals, and optionally one or more preferences associated with each of the plurality of individuals. The recommender model is trained to output a recommendation of one or more products and/or one or more routines for each of the plurality of individuals. The computing instructions of the imaging app, when executed by the one or more processors, cause the one or more processors to obtain a set of one or more images of a product associated with a user, the set of one or more images comprising pixel data as captured by an imaging device, and the pixel data depicting at least a portion of the product, detect, based on output of the product-based learning model inputting the pixel data, a product identifier of the product associated with the user, obtain user input including one or more personal parameters associated with the user, one or more goals associated with the user, and optionally one or more preferences associated with the user, predict one or more risk factors associated with the user, based on output of the risk factor model inputting the one or more personal parameters associated with the user and the product identifier of the product, recommend one or more routines and/or one or more products for the user, based on an output of the recommender model inputting the one or more personal parameters associated with the user, the product identifier of the product associated with the user, the one or more predicted risk factors associated with the user, the one or more goals associated with the user, and optionally the one or more preferences associated with the user, and output a feedback indication including an indication of the recommended one or more products and/or routines for the user.

In some aspects, the techniques described herein relate to a method for images of products and parameters associated with users and making product and/or routine recommendations using a digital imaging and artificial intelligence (AI)-based system. The method includes executing, on one or more processors, computing instructions of an imaging application (app), obtaining, via the imaging app, a set of one or more images of a product associated with a user, the set of one or more images comprising pixel data as captured by an imaging device, and the pixel data depicting at least a portion of the product, detecting, based on output of a product-based learning model accessible by the imaging app and trained with pixel data of a plurality of training images depicting one or more products, a product identifier of the product associated with the user by inputting the pixel data into the product-based learning model, obtaining user input including one or more personal parameters associated with the user, one or more goals associated with the user, and optionally one or more preferences associated with the user, predicting one or more risk factors associated with the user, based on output of a risk factor model accessible by the imaging app and trained with personal parameters associated with each of a plurality of individuals, and one or more products used by each of the plurality of individuals, by inputting the one or more personal parameters associated with the user and the product identifier of the product, recommending one or more routines and/or one or more products for the user, based on an output of a recommender model accessible by the imaging app and trained with the personal parameters associated with each of the plurality of individuals, the one or more products used by each of the plurality of individuals, the one or more predicted risk factors associated with each of the plurality of individuals, one or more goals associated with each of the plurality of individuals, and optionally one or more preferences associated with each of the plurality of individuals, by inputting the one or more personal parameters associated with the user, the product identifier of the product associated with the user, the one or more predicted risk factors associated with the user, the one or more goals associated with the user, and optionally the one or more preferences associated with the user, and outputting a feedback indication including an indication of the recommended one or more products and/or routines for the user.

In some aspects, the techniques described herein relate to a tangible, non-transitory computer-readable medium storing instructions for analyzing images of products and parameters associated with users and making product and/or routine recommendations. When executed by one or more processors, these instructions cause the one or more processors to detect, based on product data, a product identifier of a product associated with a user; obtain, by an imaging application (app), a set of one or more images of the product, the set of one or more images comprising pixel data as captured by an imaging device, and the pixel data depicting at least a portion of the product; generate, based on output of a product-based learning model, a first analysis comprising a product comparison comparing the product as depicted in pixel data to a database of products, wherein the product-based learning model is trained with pixel data of a plurality of training images depicting one or more products, the product-based learning model trained to output product predictions of one or more product identifiers corresponding to the one or more products based on the pixel data; obtain user input including one or more personal parameters associated with the user, one or more goals associated with the user, and optionally one or more preferences associated with the user; predict, based on output of a risk factor model, one or more risk factors associated with the user, wherein the risk factor model is trained with personal parameters associated with each of a plurality of individuals, and one or more products used by each of the plurality of individuals, the risk factor model trained to output one or more predicted risk factors based on the personal parameters and the product identifier of the product; recommend, based on an output of a recommender model, one or more routines and/or one or more products for the user, wherein the recommender model is trained with the personal parameters associated with each of the plurality of individuals, the one or more products used by each of the plurality of individuals, the one or more predicted risk factors associated with each of the plurality of individuals, one or more goals associated with each of the plurality of individuals, and optionally one or more preferences associated with each of the plurality of individuals, the recommender model trained to output a recommendation of one or more products and/or routines based on the personal parameters, the product identifier, the predicted risk factors, the goals, and optionally the preferences of the user; and output, based on the recommendation, a feedback indication including an indication of the recommended one or more products and/or routines for the user.

In accordance with the above, and with the disclosure herein, the present disclosure includes improvements in computer functionality or in improvements to other technologies at least because the disclosure describes that, e.g., an imaging server, or otherwise computing device (e.g., a user computer device), is improved where the intelligence or predictive ability of the server or computing device is enhanced by a trained (e.g., machine learning trained) product-based learning model, risk factor model, and recommender model. These models, executing on the imaging server or computing device, are able to more accurately identify, based on pixel data of various oral care products, a feedback indication including an indication of the recommended one or more products and/or routines for the user. That is, the present disclosure describes improvements in the functioning of the computer itself or “any other technology or technical field” because an imaging server or user computing device is enhanced with a plurality of training images (e.g., 10,000 s of training images and related pixel data as feature data) to accurately predict, detect, classify, or determine pixel data of a user-specific images, such as newly provided user images. This improves over the prior art at least because existing systems lack such predictive or classification functionality and are simply not capable of accurately analyzing user-specific images to output a predictive result based on a product identifiable within the pixel data and parameters associated with the user (including user oral care concerns and/or oral care goals, as well as oral care preferences in some cases).

For similar reasons, the present disclosure relates to improvements to other technologies or technical fields at least because the present disclosure describes or introduces improvements to computing devices in the oral care field, whereby the product-based learning model, risk factor model, and recommender model executing on the imaging device(s) or computing devices, improves the field of oral care product identification, oral care, oral hygiene, and/or oral care product recommendation and efficacy related thereto, with digital and/or artificial intelligence based analysis of product images to output a predictive result based on a product identifiable within the pixel data and the parameters associated with the user (including user oral care concerns and/or oral care goals, as well as oral care preferences in some cases).

In addition, the present disclosure relates to improvements to other technologies or technical fields at least because the present disclosure describes or introduces improvements to computing devices in the oral care product identification and recommendation field, whereby the trained product-based learning model, risk factor model, and recommender model executing on the imaging device(s) or computing device(s) improve the underlying computer device (e.g., imaging server(s) and/or user computing device), where such computer devices are made more efficient by the configuration, adjustment, adaptation, and/or otherwise update of a given machine-learning network architecture. For example, in some embodiments, fewer machine resources (e.g., processing cycles or memory storage) may be used by decreasing computational resources by decreasing machine-learning network architecture, including by reducing depth, width, image size, or other machine-learning based dimensionality requirements. Such reduction frees up the computational resources of an underlying computing system, thereby making it more efficient.

Still further, the present disclosure relates to improvement to other technologies or technical fields at least because the present disclosure describes or introduces improvements to computing devices in the field of security, where images of products are preprocessed (e.g., cropped or otherwise modified) to define extracted or depicted product portions of a product without depicting personal identifiable information (PII) of a user. For example, cropped or redacted portions of an image of a product may be used by the product-based learning model described herein, which eliminates the transmission of images that may include users using such products across a computer network (where such images may be susceptible of interception by third parties). Such features provide a security improvement, i.e., where the removal of PII (e.g., facial features) provides an improvement over prior systems because cropped or redacted images, especially ones that may be transmitted over a network (e.g., the Internet), are more secure without including PII information of a user. Accordingly, the systems and methods described herein operate without the non-essential information, which provides an improvement, e.g., a security improvement, over prior systems. In addition, the use of cropped images, at least in some embodiments, allows the underlying system to store and/or process smaller data size images, which results in a performance increase to the underlying system as a whole because the smaller data size images require less storage memory and/or processing resources to store, process, and/or otherwise manipulate by the underlying computer system.

In addition, the present disclosure includes specific features other than what is well-understood, routine, conventional activity in the field, or adding unconventional steps that confine the claim to a particular useful application, e.g., digital imaging and AI-based systems and methods for analyzing images of oral care products and parameters associated with the user (including user oral care concerns and/or oral care goals, as well as oral care preferences in some cases) and making recommendations, which may include, for example, analyzing products, oral care conditions, oral care goals, preferences, and other parameters associated with a user in real-time or near real-time to provide feedback depicted as augmented reality (AR) based data overlaid or superimposed with a recommended product or routine over one or more images (e.g., a video).

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates an example digital imaging and AI-based configured to analyze images of products and parameters associated with users and make product and/or routine recommendations, in accordance with various embodiments disclosed herein.

FIG. 2 illustrates an example image and its related pixel data that may be used for training and/or implementing a product-based learning model, in accordance with various embodiments disclosed herein.

FIG. 3 illustrates an example digital imaging and AI-based method for analyzing images of products and parameters associated with users and making product and/or routine recommendations, in accordance with various embodiments disclosed herein.

FIG. 4 illustrates an example digital imaging and AI-based method for analyzing images of products and parameters associated with users that are input by users and making product and/or routine recommendations, in accordance with various embodiments disclosed herein.

FIG. 5 illustrates an example user interface as rendered on a display screen of a user computing device in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION OF THE INVENTION

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral product composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral product compositions include dentifrice, toothpaste, tooth gel, subgingival gel, emulsion, mouth rinse, mousse, foam, mouth spray, lozenge, chewable tablet, chewing gum, tooth whitening strips, floss and floss coatings, breath freshening dissolvable strips, unit-dose composition, fibrous composition, or denture care or adhesive product. The oral product composition may also be incorporated onto strips or films for direct application or attachment to oral surfaces, such as tooth whitening strips. Examples of emulsion compositions include the emulsions compositions of U.S. Pat. No. 11,147,753, jammed emulsions, such as the jammed oil-in-water emulsions of U.S. Pat. No. 11,096,874. Examples of unit-dose compositions include the unit-dose compositions of U.S. Patent Application Publication No. 2019/0343732.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health, e.g., toothpaste, mouth rinse, floss, toothpicks, spray, water irrigator, massager.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, the singular “an oral product composition” or “a bleaching agent” may also include the plural unless the context specifically states otherwise.

Several types of ranges are disclosed in relation to embodiments of the present invention. When a range of any type is disclosed or claimed, the intent is to disclose or claim individually each possible number that such a range could reasonably encompass, including end points of the range as well as any sub-ranges and combinations of sub-ranges encompassed therein.

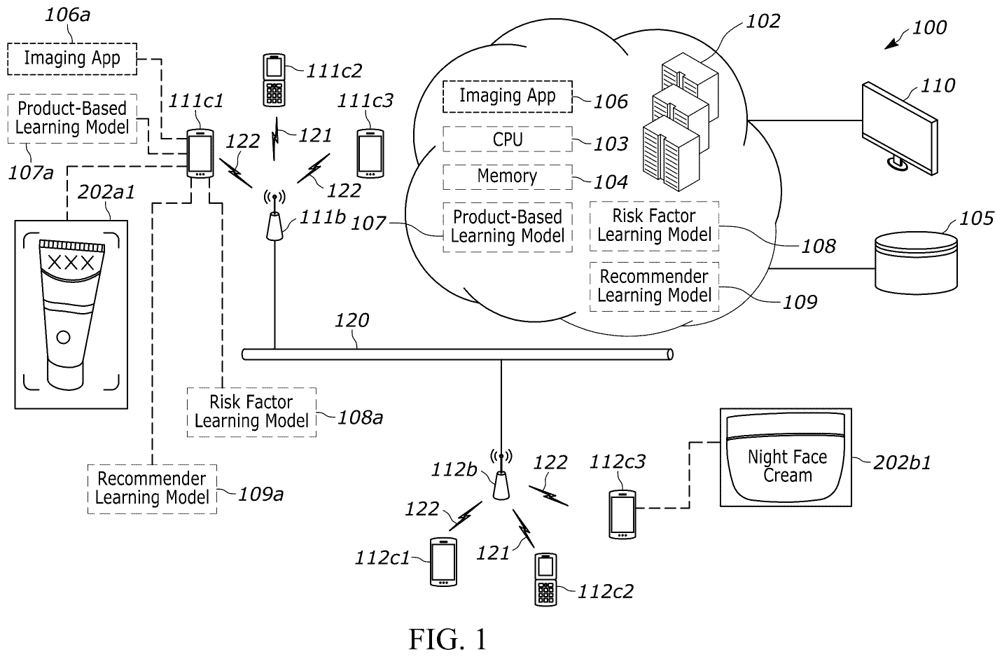

FIG. 1 illustrates an example digital imaging and artificial intelligence (AI)-based system 100 configured to analyze images of products and parameters associated with users in order to generate recommendations for users, in accordance with various embodiments disclosed herein. In the example embodiment of FIG. 1, digital imaging and AI-based system 100 includes server 102, which may comprise one or more computer servers. In various embodiments server 102 may comprise multiple servers, which may comprise multiple, redundant, or replicated servers as part of a server farm. In still further embodiments, server 102 may be implemented as cloud-based servers, such as a cloud-based computing platform. For example, imaging server 102 may be any one or more cloud-based platform(s) such as MICROSOFT AZURE, AMAZON AWS, or the like.

Server 102 may include one or more processor 103 (i.e., CPU(s)) as well as one or more computer memories 104. In various embodiments, server 102 may be referred to herein as “imaging server(s).”

Memory 104 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memory 104 may store an operating system (OS) (e.g., Microsoft Windows, Linux, UNIX, etc.) capable of facilitating the functionalities, apps, methods, or other software as discussed herein. Memory 104 may store an imaging application (app) 106, as well as a product-based learning model 107, a risk factor learning model 108, and/or a recommender learning model 109, each of which may comprise artificial intelligence-based models, such as machine learning models, trained on various images (e.g., images 202a1 and/or 202b1), as described herein. Additionally, or alternatively, the product-based learning model 107, the risk factor learning model 108, and/or the recommender learning model 109 may also be stored in database 105, which is accessible or otherwise communicatively coupled to imaging server 102. In addition, memories 104 may also store machine readable instructions, including any of one or more application(s) (e.g., an imaging application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, at least some of the applications, software components, or APIs may be, include, otherwise be part of, a machine learning model or component, such as the product-based learning model 107, the risk factor learning model 108, and/or the recommender learning model 109, where each may be configured to facilitate their various functionalities discussed herein. It should be appreciated that one or more other applications may be envisioned and that are executed by the processor 103.

The processor 103 may be connected to the memories 104 via a computer bus responsible for transmitting electronic data, data packets, or otherwise electronic signals to and from the processor 103 and memories 104 in order to implement or perform the machine-readable instructions, methods, processes, elements or limitations, as illustrated, depicted, or described for the various flowcharts, illustrations, diagrams, figures, and/or other disclosure herein.

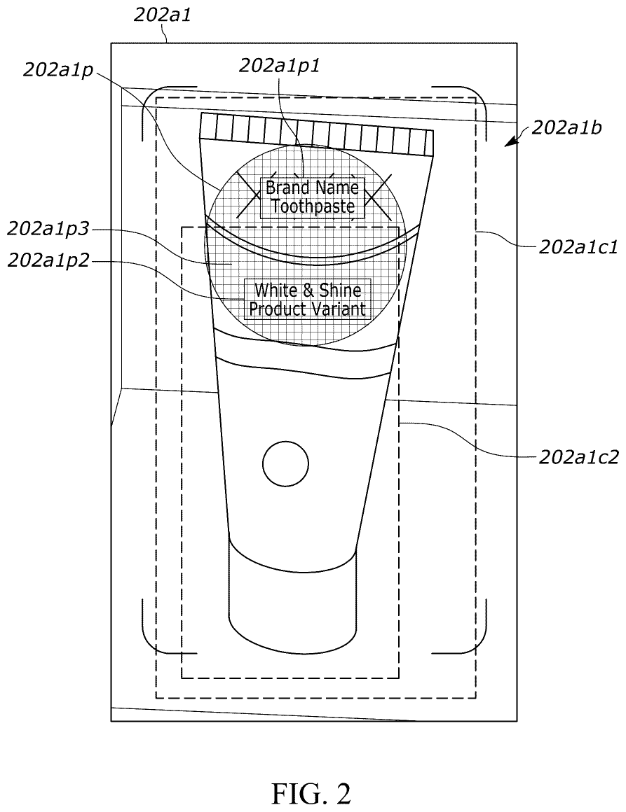

Processor 103 may interface with memory 104 via the computer bus to execute an operating system (OS). Processor 103 may also interface with the memory 104 via the computer bus to create, read, update, delete, or otherwise access or interact with the data stored in memories 104 and/or the database 105 (e.g., a relational database, such as Oracle, DB2, MySQL, or a NoSQL based database, such as MongoDB). The data stored in memories 104 and/or database 105 may include all or part of any of the data or information described herein, including, for example, training images and/or user images (e.g., including any one or more of images 202a1 and/or 202b1, and/or zoomed, cropped, and/or segmentation related images, for example as shown at FIG. 2), and/or other images and/or information of products, or other information or data as otherwise described herein.

Imaging server 102 may further include a communication component configured to communicate (e.g., send and receive) data via one or more external/network port(s) to one or more networks or local terminals, such as computer network 120 and/or terminal 110 (for rendering or visualizing) described herein. In some embodiments, imaging server 102 may include a client-server platform technology such as ASP.NET, Java J2EE, Ruby on Rails, Node.js, a web service or online API, responsive for receiving and responding to electronic requests. The imaging server 102 may implement the client-server platform technology that may interact, via the computer bus, with the memories 104 (including the application(s), component(s), API(s), data, etc. stored therein) and/or database 105 to implement or perform the machine readable instructions, methods, processes, elements or limitations, as illustrated, depicted, or described for the various flowcharts, illustrations, diagrams, figures, and/or other disclosure herein.

In various embodiments, the imaging server 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some embodiments, computer network 120 may comprise a private network or local area network (LAN). Additionally, or alternatively, computer network 120 may comprise a public network such as the Internet.

Imaging server 102 may further include or implement an operator interface configured to present information to an administrator or operator and/or receive inputs from the administrator or operator. As shown in FIG. 1, an operator interface may provide a display screen (e.g., via terminal 110). Imaging server 102 may also provide I/O components (e.g., ports, capacitive or resistive touch sensitive input panels, keys, buttons, lights, LEDs), which may be directly accessible via, or attached to, imaging server 102 or may be indirectly accessible via or attached to terminal 110. According to some embodiments, an administrator or operator may access the server 102 via terminal 110 to review information, make changes, input training data or images, initiate training of the product-based learning model 107, the risk factor learning model 108, and/or the recommender learning model 109, and/or perform other functions.

As described herein, in some embodiments, imaging server 102 may perform the functionalities as discussed herein as part of a “cloud” network or may otherwise communicate with other hardware or software components within the cloud to send, retrieve, or otherwise analyze data or information described herein.

In general, a computer program or computer based product, application, or code (e.g., the model(s), such as AI models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor 103 (e.g., working in connection with the respective operating system in memories 104) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C #, Objective-C, Java, Scala, ActionScript, JavaScript, HTML, CSS, XML, etc.).

As shown in FIG. 1, imaging server(s) 102 are communicatively connected, via computer network 120 to the one or more user computing devices 111c1-111c3 and/or 112c1-112c3 via base stations 111b and 112b. In some embodiments, base stations 111b and 112b may comprise cellular base stations, such as cell towers, communicating to the one or more user computing devices 111c1-111c3 and 112c1-112c3 via wireless communications 121 based on any one or more of various mobile phone standards, including NMT, GSM, CDMA, UMMTS, LTE, 5G, or the like. Additionally, or alternatively, base stations 111b and 112b may comprise routers, wireless switches, or other such wireless connection points communicating to the one or more user computing devices 111c1-111c3 and 112c1-112c3 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c3 and/or 112c1-112c3 may comprise mobile devices and/or client devices for accessing and/or communications with imaging server 102. Such mobile devices may comprise one or more mobile processor(s) and/or an imaging device for capturing images, such as images as described herein (e.g., any one or more of images 202a1 and/or 202b1). In various embodiments, user computing devices 111c1-111c3 and/or 112c1-112c3 may comprise a mobile phone (e.g., a cellular phone), a tablet device, a personal data assistance (PDA), or the like, including, by non-limiting example, an APPLE IPHONE or IPAD device or a GOOGLE ANDROID based mobile phone or table.

In various embodiments, the one or more user computing devices 111c1-111c3 and/or 112c1-112c3 may implement or execute an operating system (OS) or mobile platform such as Apple's iOS and/or Google's Android operation system. Any of the one or more user computing devices 111c1-111c3 and/or 112c1-112c3 may comprise one or more processors and/or one or more memories for storing, implementing, or executing computing instructions or code, e.g., a mobile application or a home or personal assistant application, as described in various embodiments herein. As shown in FIG. 1, the imaging app 106a, the product-based learning model 107a, the risk factor learning model 108a, and/or the recommender learning model 109a as described herein, or at least portions thereof, may also be stored locally on a memory of a user computing device (e.g., user computing device 111c1). In some aspects, the imaging app 106a, the product-based learning model 107a, the risk factor learning model 108a, and/or the recommender learning model 109a as installed on a computing device may comprise a same imaging app 106, the product-based learning model 107, the risk factor learning model 108, and/or the recommender learning model 109 as installed on server 102. Additionally, or alternatively, the imaging app 106a, the product-based learning model 107a, the risk factor learning model 108a, and/or the recommender learning model 109a may comprise a portion of the imaging app 106, the product-based learning model 107, the risk factor learning model 108, and/or the recommender learning model 109 as installed on server 102, where such respective models can communicate with each other across computer network 120. Further, it is to be understood that in some aspects, the imaging app, the product-based learning model, the risk factor learning model, and/or the recommender learning model may be installed wholly at user computing device, wholly at server 102, or partially on user computing device and partially on server 102 where communication between the imaging app 106a and the imaging app 106, the product-based learning model 107a and the product-based learning model 107, the risk factor learning model 108a and the risk factor learning model 108, and/or the recommender learning model 109a and the recommender learning model 109, occurs through computer network 120. Generally, when a given model or app is referred to herein, it refers respectively to one or both of the given app or model, whether operating alone at the sever or computing device, or whether communicating over computer network 120.

User computing devices 111c1-111c3 and/or 112c1-112c3 may comprise a wireless transceiver to receive and transmit wireless communications 121 and/or 122 to and from base stations 111b and/or 112b. In various embodiments, pixel-based images (e.g., images 202a1 and/or 201b1) may be transmitted via computer network 120 to imaging server 102 for training of model(s) (e.g., the product-based learning model, the risk factor learning model, and/or the recommender learning model) and/or imaging analysis as described herein.

In addition, the one or more user computing devices 111c1-111c3 and/or 112c1-112c3 may include an imaging device (e.g., a camera) and/or digital video camera for capturing or taking digital images and/or frames (e.g., which can be any one or more of images 202a1 and/or 202b1). Each digital image may comprise pixel data for training or implementing model(s), such as AI or machine learning models, as described herein. For example, an imaging device and/or digital video camera of, e.g., any of user computing devices 111c1-111c3 and/or 112c1-112c3, may be configured to take, capture, or otherwise generate digital images (e.g., pixel-based images 202a1 and/or 202b1) and, at least in some embodiments, may store such images in a memory of a respective user computing devices. Additionally, or alternatively, such digital images may also be transmitted to and/or stored on memory 104 and/or database 105 of server 102.

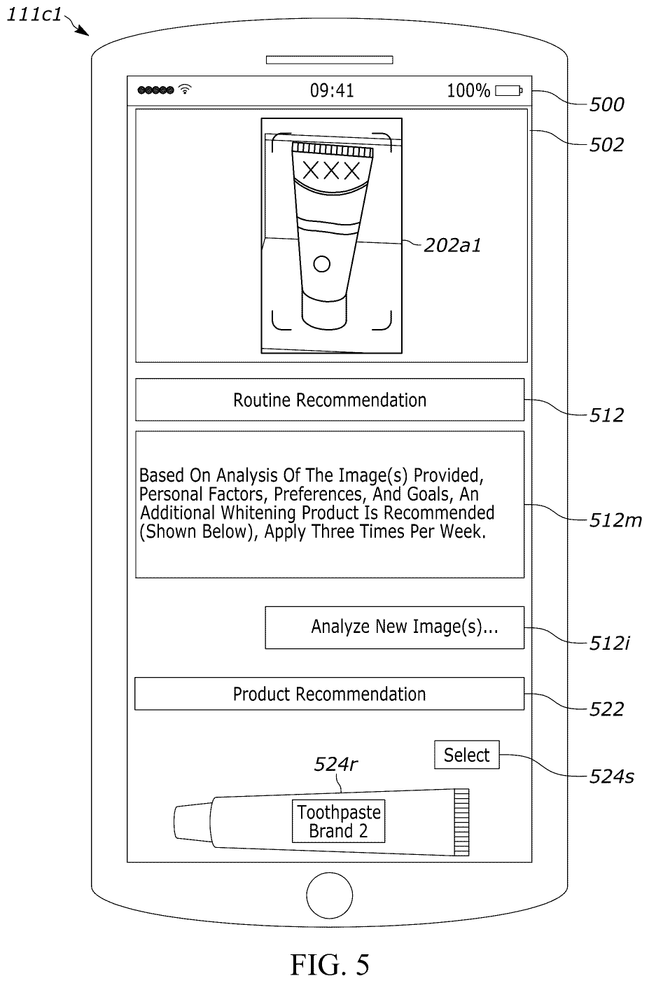

Still further, each of the one or more user computer devices 111c1-111c3 and/or 112c1-112c3 may include a display screen for displaying graphics, images, text, product(s), data, pixels, features, and/or other such visualizations or information as described herein. In various embodiments, graphics, images, text, product(s), data, pixels, features, and/or other such visualizations or information may be received from imaging server 102 for display on the display screen of any one or more of user computer devices 111c1-111c3 and/or 112c1-112c3. Additionally, or alternatively, a user computer device, e.g., as described herein for FIG. 5, may comprise, implement, have access to, render, or otherwise expose, at least in part, an interface or a guided user interface (GUI) for displaying text and/or images on its display screen.

In some embodiments, computing instructions and/or applications executing at the server (e.g., server 102) and/or at a mobile device (e.g., mobile device 111c1) may be communicatively connected for analyzing pixel data of an image of a product to generate or otherwise output a feedback indication(s) including an identification of the product and/or an indication of one or more recommended products and/or routines for the user, as described herein. For example, one or more processors (e.g., processor 103) of server 102 may be communicatively coupled to a mobile device via a computer network (e.g., computer network 120). In such embodiments, an imaging app may comprise a server app portion configured to execute on the one or more processors of the server (e.g., server 102) and a mobile app portion configured to execute on one or more processors of the mobile device (e.g., any of one or more user computing devices 111c1-111c3 and/or 112c1-112c3). In such embodiments, the server app portion is configured to communicate with the mobile app portion. The server app portion or the mobile app portion may each be configured to implement, or partially implement, one or more of: (1) obtaining a set of one or more images of a product associated with a user, the set of one or more images comprising pixel data as captured by an imaging device, and the pixel data depicting least a portion of the product; (2) detecting, based on output of the product-based learning model inputting the pixel data, a product identifier of the product associated with the user; (3) obtaining user input including one or more personal parameters associated with the user, one or more goals associated with the user, and optionally one or more preferences associated with the user; (4) predicting one or more risk factors associated with the user, based on output of the risk factor model inputting the one or more personal parameters associated with the user and the product identifier of the product; (5) recommending one or more routines and/or one or more products for the user, based on an output of the recommender model inputting the one or more personal parameters associated with the user, the product identifier of the product associated with the user, the one or more predicted risk factors associated with the user, the one or more goals associated with the user, and optionally the one or more preferences associated with the user; and/or (6) outputting a feedback indication including an indication of the recommended one or more products and/or routines for the user.

FIG. 2 illustrates an example image 202a1 and its related pixel data that may be used for training and/or implementing a product-based learning model, in accordance with various embodiments disclosed herein. In various embodiments, as shown for FIG. 2, image 202a1 may be an image captured by a user. More generally, image 202a1 (as well as image 202b1) may be transmitted to server 102 via computer network 120, as shown for FIG. 1. It is to be understood that such images may be captured by the users themselves or, additionally or alternatively, others, where such images are used and/or transmitted on behalf of a user.

Digital images, such as non-limiting example images 202a1 and/or 202b1, may be collected or aggregated at imaging server 102 and may be analyzed by, and/or used to train, an AI-based model (e.g., an AI model such as the product-based learning model described herein). Each of these images may comprise pixel data comprising feature data and corresponding to product(s), product dosage(s), product implement(s), product appliance(s), background(s), and/or other features described herein. The pixel data may be captured by an imaging device of one of the user computing devices (e.g., one or more user computer devices 111c1-111c3 and/or 112c1-112c3).

With respect to digital images as described herein, pixel data (e.g., pixel data 202a1p of FIG. 2) comprises individual points or squares of data within an image, where each point or square represents a single pixel (e.g., each of pixel 202a1p1, pixel 202a1p2, and pixel 202a1p3) within an image. Each pixel may be at a specific location within an image. In addition, each pixel may have a specific color (or lack thereof). Pixel color may be determined by a color format and related channel data associated with a given pixel. For example, a popular color format is a 1976 CIELAB (also referenced herein as the “CIE L*−a*−b*” or simply “L*a*b*” color format) color format that is configured to mimic the human perception of color. Namely, the L*a*b* color format is designed such that the amount of numerical change in the three values representing the L*a*b* color format (e.g., L*, a*, and b*) corresponds roughly to the same amount of visually perceived change by a human. This color format is advantageous, for example, because the L*a*b* gamut (e.g., the complete subset of colors included as part of the color format) includes the gamuts of Red (R), Green (G), and Blue (B) (collectively RGB) and Cyan (C), Magenta (M), Yellow (Y), and Black (K) (collectively CMYK) color formats.

In the L*a*b* color format, color is viewed as point in three dimensional space, as defined by the three-dimensional coordinate system (L*, a*, b*), where each of the L* data, the a* data, and the b* data may correspond to individual color channels, and may therefore be referenced as channel data. In this three-dimensional coordinate system, the L* axis describes the brightness (luminance) of the color with values from 0 (black) to 100 (white). The a* axis describes the green or red ratio of a color with positive a* values (+a*) indicating red hue and negative a* values (−a*) indicating green hue. The b* axis describes the blue or yellow ratio of a color with positive b* values (+b*) indicating yellow hue and negative b* values (−b*) indicating blue hue. Generally, the values corresponding to the a* and b* axes may be unbounded, such that the a* and b* axes may include any suitable numerical values to express the axis boundaries. However, the a* and b* axes may typically include lower and upper boundaries that range from approximately 150 to −150. Thus, in this manner, each pixel color value may be represented as a three-tuple of the L*, a*, and b* values to create a final color for a given pixel.

As another example, a popular color format includes the red-green-blue (RGB) format having red, green, and blue channels. That is, in the RGB format, data of a pixel is represented by three numerical RGB components (Red, Green, Blue), that may be referred to as a channel data, to manipulate the color of pixel's area within the image. In some implementations, the three RGB components may be represented as three 8-bit numbers for each pixel. Three 8-bit bytes (one byte for each of RGB) may be used to generate 24-bit color. Each 8-bit RGB component can have 256 possible values, ranging from 0 to 255 (i.e., in the base 2 binary system, an 8-bit byte can contain one of 256 numeric values ranging from 0 to 255). This channel data (R, G, and B) can be assigned a value from 0 to 255 that can be used to set the pixel's color. For example, three values like (250, 165, 0), meaning (Red=250, Green=165, Blue=0), can denote one Orange pixel. As a further example, (Red=255, Green=255, Blue-0) means Red and Green, each fully saturated (255 is as bright as 8 bits can be), with no Blue (zero), with the resulting color being Yellow. As a still further example, the color black has an RGB value of (Red-0, Green-0, Blue=0) and white has an RGB value of (Red=255, Green=255, Blue=255). Gray has the property of having equal or similar RGB values, for example, (Red=220, Green=220, Blue=220) is a light gray (near white), and (Red=40, Green=40, Blue=40) is a dark gray (near black).

In this way, the composite of three RGB values creates a final color for a given pixel. With a 24-bit RGB color image, using 3 bytes to define a color, there can be 256 shades of red, and 256 shades of green, and 256 shades of blue. This provides 256×256×256, i.e., 16.7 million possible combinations or colors for 24 bit RGB color images. As such, a pixel's RGB data value indicates a degree of color or light each of a Red, a Green, and a Blue pixel is comprised of. The three colors, and their intensity levels, are combined at that image pixel, i.e., at that pixel location on a display screen, to illuminate a display screen at that location with that color. It is to be understood, however, that other bit sizes, having fewer or more bits, e.g., 10-bits, may be used to result in fewer or more overall colors and ranges.

As a whole, the various pixels, positioned together in a grid pattern (e.g., pixel data 202a1p), form a digital image or portion thereof. A single digital image can comprise thousands or millions of pixels. Images can be captured, generated, stored, and/or transmitted in a number of formats, such as JPEG, TIFF, PNG and GIF. These formats use pixels to store or represent the image.

With reference to FIG. 2, example image 202a1 illustrates a product (e.g., a tube of toothpaste) that appears in a typical location, e.g., bathroom or countertop space with the countertop and backsplash as a background or otherwise background feature. More specifically, image 202a1 comprises pixel data, including pixel data 202a1p defining a product region of product packaging (e.g., a tube) of a product (e.g., toothpaste). Pixel data 202a1p includes a plurality of pixels including pixel 202a1p1, pixel 202a1p2, and pixel 202a1p3. In example image 202a1, each of pixel 202a1p1, pixel 202a1p2, and pixel 202a1p3 are representative of features of a product defining or otherwise corresponding to product data. Generally, in various embodiments, product data or otherwise product feature data may comprise one or more features identifiable with the pixel data of a given image. Each of these features may be determined from or otherwise based on one or more pixels in a digital image (e.g., image 202a1). For example, with respect to image 202a1, pixel 202a1p1 may be a relatively white pixel (e.g., pixels with relatively high RGB values across all RGB channels) positioned within pixel data 202a1p of the product packaging of the product (e.g., toothpaste) of FIG. 2, which may be indicative of a brand name of toothpaste product as depicted in white text. The pixels may form a pattern in the shape of letters or styling indicative of the brand name.

Pixel 202a1p2 may comprise relatively red pixels (e.g., pixels with high R (red) values in RGB based channels indicating a red color) and may be indicative of a typical color associated with the brand name of the product (e.g., the brand name toothpaste product is typically associated with the color red). As a further example, pixel 202a1p2 may also be part of a pattern of pixels defining an edge of the product packaging (e.g., tube of toothpaste), which can be used to determine the shape of the product packaging, and therefore detect the product or otherwise product identifier. In some aspects, such shape, pattern, or edge may be used by a segmentation model to determine or detect what product or product identifier depicted in a given image.

As a further example, pixel 202a1p3 may comprise darker pixels (e.g., with lower values in the RGB based channels), which may be indicative of smaller font text that indicates a product variant, which defines a type of product (e.g., teeth whitening toothpaste). Such pixel features may comprise product data that may be used to train a product-based learning model (e.g., train the product-based learning model 107) to detect, based on the product data, a product or product identifier of a product.

In addition to pixels 202a1p1, 202a1p2, and 202a1p3, pixel data 202a1p includes various other pixels including remaining portions of the product packaging, including various other product data or information that may be analyzed and/or used for training of model(s), and/or analysis by used of already trained models, such as product-based learning model 108 and/or product-based learning model 108a as described herein. For example, pixel data 202a1p further includes pixels representative of features of SKUs, barcodes, QR codes, product text and/or other features identified in the pixel data and/or at a particular location in the image, where such pixels comprise unique identifiable features, which provides training information for detecting, based on such product data as identifiable in the pixel data, a product identifier of a product as described herein.

In addition, digital images of a products, as described herein, may depict various product features, which may be used to train a product-based learning model across a variety of different products having a variety of different product features. For example, as illustrated for images 202a1 and 202b1, the product features of these different can be different, where, for example the products have different shapes, labels, SKUs, barcodes, QR codes, brand names, product names, and the like.

A digital image, such as a training image, an image as submitted by users, or otherwise a digital image (e.g., any of images 202a1 and/or 202b1), may be or may comprise a cropped image.

Generally, a cropped image is an image with one or more pixels removed, deleted, or hidden from an originally captured image. In some aspects, each image of the one or more of the plurality of training images e.g., any of images 202a1 and/or 202b1) or the image of a product and/or product dosage comprises at least one cropped image depicting the product or product dosage having a given feature. For example, with reference to FIG. 2, cropped portion 202a1c1 represents a first cropped portion of image 202a1 that removes portions of the background 202a1b or non-product features (outside of cropped portion 202a1c1) that may not include readily identifiable regions that have a product and/or product features. As a further example, cropped portion 202a1c2 represents a second cropped portion of image 202a1 that removes further portions of the image (outside of cropped portion 202a1c2) that includes additional background or non-product features compared to the cropped portion 202a1c1, and therefore reduces the amount of pixels and data that the give system (e.g., system 100) must store or otherwise analyze as training data.

In various embodiments, analyzing and/or use of cropped images for training yields improved accuracy of a training the learning models (e.g., a product-based learning model). It also improves the efficiency and performance of the underlying computer system in that such system processes, stores, and/or transfers smaller size digital images. Still further, images may be sent as cropped or that otherwise include extracted or depicted product and/or product dosage without depicting personal identifiable information (PII) of a user. In some aspects, each image of a plurality of training images may comprise at least one cropped image removing at least a portion of PII of a user. For example, a cropping algorithm automatically crops each item in the image (if more than one is presented), to check and crop out human/facial accidental images (e.g., a mirror reflection) and remove such PII data. Such cropped images provide a security improvement, i.e., where the removal of PII provides an improvement over prior systems because cropped or redacted images, especially ones that may be transmitted over a network (e.g., the Internet), are more secure without including PII information of a user. Importantly, the systems and methods described herein may operate without the need for such non-essential information and thus is able to operate with smaller data size images, which provides an improvement, e.g., a security and a performance improvement, over conventional systems. Moreover, while FIG. 2 may depict and describe a cropped image, it is to be understood, however, that other image types including, but not limited to, original, non-cropped images (e.g., original image 202a1) and/or other types/sizes of cropped images may be used or substituted as well.

In various embodiments, digital images (e.g., images 202a1 and/or 202b1), whether used as training images depicting products, or used as images depicting product dosages by specific users, may comprise multiple angles or perspectives. That is, each image of the one or more of the plurality of training images or the image of a user may comprise multiple angles or perspectives depicting products, product implements, product appliances, and/or other features as described herein. The multiple angles or perspectives may include different views, angular positions, zoomed positions of the user and/or backgrounds, lighting conditions, and/or otherwise environments in which a given product, product implement, and/or product appliance is positioned within a given image. For example, FIG. 1 includes images (e.g., 202a1 and 202b1) that depict products, product packaging,, and/or product implements that were captured using different lighting conditions (e.g., visible, UV) at different angles. Such images may be used for training AI models (e.g., a product-based learning model), or for analysis, and/or feedback indication(s) including an identification of the product and/or an indication of one or more recommended products and/or routines for the user, as described herein.

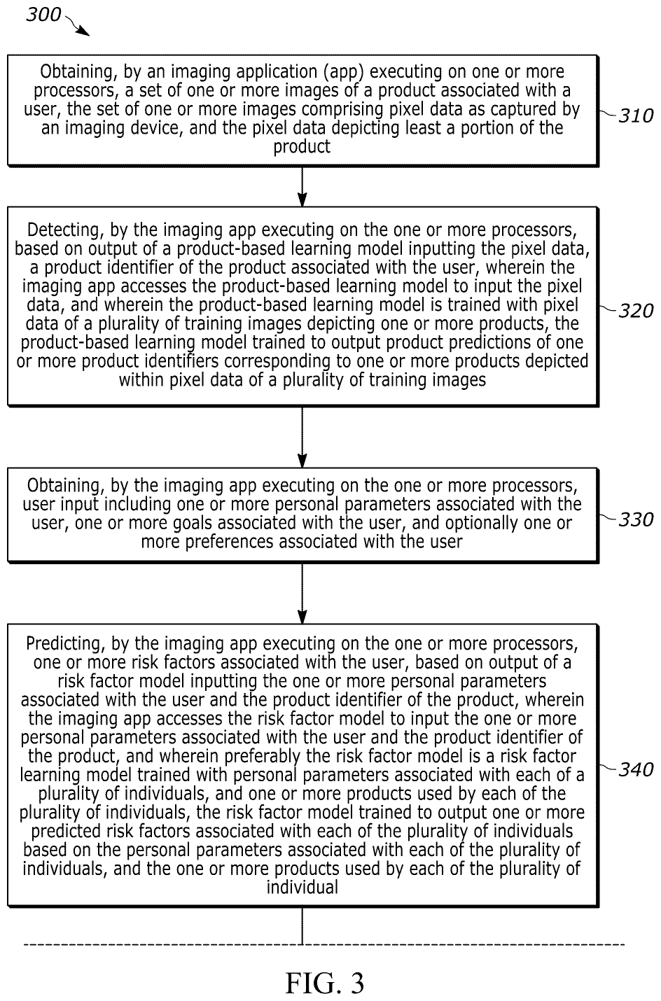

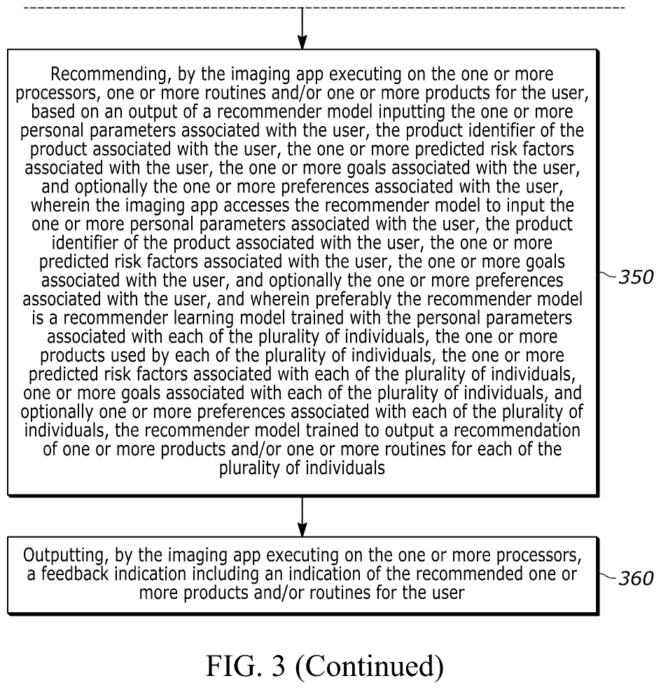

FIG. 3 illustrates an example digital imaging and AI-based method 300 for analyzing images of products and parameters associated with users in order to generate recommendations for users, in accordance with various embodiments disclosed herein. Method 300 may be implemented by one or more processors, including processor 103, a processor of a mobile device (e.g., such as computing device 111c1), or multiple processors, such as processor 103 and a processor of a mobile device (e.g., such as computing device 111c1) communicating across computer network 120.

At block 310, the method 300 comprises obtaining, by one or more processors, a set of one or more images of the product, the set of one or more images comprising pixel data as captured by an imaging device, and the pixel data depicting at least a portion of the product.

At block 320, the method 300 comprises detecting, by one or more processors based on the one or more images (i.e., based on the pixel data), a product identifier of the product. In various aspects, a product may comprise an oral product composition, an oral implement, or an oral appliance. However, it should be understood, that the disclosure herein could relate to other products, including non-oral products or otherwise different products, such as skin care products, shaving products, and/or other consumer products.

In some aspects, the detection of a given product identifier may be based on product data comprising information provided to the imaging app (e.g., imaging app 106) by a user. For example, in some aspects, a user may input, into a graphic user interface (GUI) (e.g., as described herein for FIG. 5) an identifier, description, or otherwise indication of what product is being used. The user may also input other information, including, by way of non-limiting example, a product implement and/or product appliance is being used.

Still further, in various aspects, a product identifier can be submitted (e.g., to imaging app 106 and/or server 102) as an input to look up or link to additional data defining the product. Such additional data may include a formula specification of the product (e.g., the chemical formula of a toothpaste or mouthwash), traits of the products (e.g., the texture or viscosity of a cream, toothpaste, gel, etc.), the packaging data of the product (e.g., the shape, color, markings, or other characteristics of a given product, and/or clinical indications of the product (e.g., the active ingredient of a product, the expected effect of the product at certain amounts or dosages, and/or the expected efficacy of a product at certain amounts or dosages). More generally, with a unique product identified (e.g., by way of detecting the given product identifier), imaging app 106 and/or server 102 can look-up and link to other data sources where different information is provided about the product. This includes, but is not limited to, formulation specifications, product traits, packaging traits, clinical indications, etc. This additional data can comprise data package, or otherwise meta data, about a given product that augments data collection and identification efforts of the product and allows identification of the product with little or no input by the user. In addition, such additional data (e.g., data package and/or meta data) can be provided, e.g., via input by a user or programmatically upon detection within pixel data, to assist with identifying related products, e.g., a toothpaste related product, once identified, can be used to detect a product implement such as a toothbrush and/or a product appliances, such as dentures. Such cross identification can be implemented, for example, where such products and product implements/product appliances are correlated product (e.g., typically used together). In addition, such additional data (e.g., data package and/or meta data) may also be provided to the product-based learning model (e.g., product-based learning model 107 and/or product-based learning model 107a) as described herein to improve its output and/or prediction related to the feedback it provides regarding product identification, for example, as described herein.

Additionally, or alternatively, in some aspects a product identifier may be as detected by the product-based learning model. For example, with reference for FIG. 1, method 300 may implement product-based learning model 107 and/or product-based learning model 107a. The product-based learning model (e.g., product-based learning model 107 and/or product-based learning model 107a) is an image (or video) based model or algorithm that allows a user to capture a digital image of one or more product compositions, product packages, product implements, product appliances, and/or other features as described herein, each of which can be in an isolated space or in a broad context space (e.g., a bathroom counter surrounded by other non-related products/implements/devices). Further, the image(s) may comprise pixel data of at least a portion of a product dosage, a product implement and/or a product application, for example, as described herein.

In various aspects, the one or more processors may comprise processor 103 of server 102. Additionally, or alternatively, the one or more processors may comprise a processor of a mobile device (e.g., computing device 111c1). Images, as used with method 300, and more generally as described herein, are pixel-based images as captured by an imaging device (e.g., an imaging device of user computing device 111c1). In some embodiments an image may comprise or refer to a plurality of images such as a set of images (e.g., frames) as collected using a digital video camera.

Frames comprise consecutive images defining motion, and can comprise a movie, a video, or the like.

In various aspects, the product-based learning model (e.g., product-based learning model 107 and/or product-based learning model 107a) can comprise an artificial intelligence (AI) based model trained with at least one AI algorithm. Training of the product-based learning model (e.g., product-based learning model 107 and/or product-based learning model 107a) involves image analysis of the training images to configure weights of the product-based learning model, and its underlying algorithm (e.g., machine learning or artificial intelligence algorithm) used to predict and/or classify additional images. For example, in various embodiments herein, generation of the product-based learning model involves training the product-based learning model with the plurality of training images (e.g., images 202a1 and/or 202b1), each of which may depict a plurality of products, product implements, product appliances, background features, and/or other features described herein, where each of the training images comprise pixel data and depict features of the product and/or these product related features.

In some aspects, one or more processors of a server or a cloud-based computing platform (e.g., imaging server 102) may receive the plurality of training images (e.g., images 202al and/or 202b1) of the plurality of products via a computer network (e.g., computer network 120). In such embodiments, the server and/or the cloud-based computing platform may train the product-based learning model (e.g., product-based learning model 107 and/or product-based learning model 107a) with the pixel data of the plurality of training images. Additionally, in some aspects, the product-based learning model may be further trained with additional data (e.g., packaged data or meta data), such as text data or product information data including, for example, product brand names, slogans, product labeling, SKUs, or otherwise as described herein as related to a product, product implement, ad/or applications. In such aspects, predictions or otherwise output, as generated by the product-based learning model (e.g., product-based learning model 107 and/or product-based learning model 107a), may be based on such text data or product data.

In various aspects, a machine learning imaging model, such as the product-based learning model (e.g., product-based learning model 107 and/or product-based learning model 107a), may be trained using a supervised or unsupervised machine learning program or algorithm. The machine learning program or algorithm may employ a neural network, which may be a convolutional neural network, a vision transformer, a deep learning neural network, or a combined learning algorithm or program that learns based on features or feature datasets (e.g., pixel data) in a particular areas of the image of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on imaging server 102. For example, libraries may include the TENSORFLOW based library, the PYTORCH library, and/or the SCIKIT-LEARN Python library.

Machine learning may involve identifying and recognizing patterns in existing data (such as identifying features of a given product, product implement, and/or product appliance, in the pixel data of image as described herein) in order to facilitate making predictions, classifications, or identification for subsequent data (such as using the model on new pixel data of a new image in order to detect a product identifier within the pixel data and/or determine or generate a feedback indication designed to address at least one feature identifiable within the pixel data comprising the dosage of the product).

Machine learning model(s), such as the product-based learning model (e.g., product-based learning model 107 and/or product-based learning model 107a) as described herein for some embodiments, may be created and trained based upon example data (e.g., training data and related pixel data) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

Supervised learning and/or unsupervised machine learning may also comprise retraining, relearning, or otherwise updating models with new, or different, information, which may include information received, ingested, generated, or otherwise used over time. The disclosures herein may use one or both of such supervised or unsupervised machine learning techniques.

In various aspects, the product-based learning model (e.g., product-based learning model 107 and/or product-based learning model 107a) may be trained, by one or more processors (e.g., one or more processor (e.g., processor 103) of server 102 and/or processors of a computer user device, such as a mobile device) with the pixel data of a plurality of training images (e.g., any of images 202al and/or 202b1). In various aspects, the product-based learning model is configured or otherwise trained to output product predictions of one or more product identifiers corresponding to the one or more products depicted within the pixel data of the plurality of training images. For example, in some aspects, the one or more product identifiers are based on prediction(s) of the product-based learning and detection model, where the model predictions predict, with a degree of predictive accuracy, how likely it is that a given product (and/or product implement and/or application) depicted in one or more images is actually the product (and/or product implement and/or application) actually depicted. The prediction(s) may be based on pixel data and features identified in images as described, for example, by FIG. 2 herein.

In various aspects, product-based learning model (e.g., product-based learning model 107 and/or product-based learning model 107a) is accessible by imaging app 106 and/or imaging app 106a, and can be implemented on a computing device (e.g., computing device 111c1) and/or server 102, where computing instructions, implementing on, one or more processors of the computing device (e.g., computing device 111c1) and/or server 102 obtain a set of one or more images of a product. In such aspects, the product data comprises the set of one or more images of the product. Further, the set of one or more images (e.g., any of images 202a1 and/or 202b1) of the product comprises pixel data of the product as captured by an imaging device. The pixel data of the product may depict least a portion of the product, product implement, product appliance, or other features described herein.

The product-based learning model (e.g., product-based learning model 107 and/or product-based learning model 107a) may then detect, based on output (e.g., a product prediction output by the model) of the product-based learning model inputting the pixel data of the product, the product identifier of the product. In various aspects, one or more product identifiers as output by the product-based learning model may be based one or more features identifiable within the pixel data of the plurality of training images. Such features may comprise, by way of none limiting example, pixel data defining or forming pixel patterns within image(s) defining a product category (e.g., oral care or skin care) of the one or more products; a product brand of the one or more products; a product variant (e.g., a specific type or formulation) of the one or more products; a product form (e.g., a texture, viscosity, pattern) of the one or more products, and/or a product packaging (e.g., a shape, color, or size) of the one or more products. For example, the product-based learning model may detect a franchise or category of a product, which may comprise a segment of a product market that the product belongs to (e.g., oral care such as toothbrushes, oral care-implements, and/or skin care-facial cleansers). As a further example, the product-based learning model may detect a brand of a product this is determined from pixel data of text or logo image processing from the image. As a further example, the product-based learning model may detect a product variant, which may be determined from analyzed text detected in the image. As a further example, the product-based learning model may detect a form of the product, which may comprise, for example, one or more physical properties of the product (e.g., a liquid, a gel, a powder, a toothbrush, etc.). As a further example, the product-based learning model may detect the product packaging of a product, which may comprise, e.g., a tube, a box, a bag, etc.). The product-based learning model may be trained to detect one or all of these the features in the pixel data to narrow and predict a unique product, product implement, and/or product appliance.

In some aspects, the product-based learning model (e.g., product-based learning model 107 and/or product-based learning model 107a) may be trained to output a product prediction defining a percentage accuracy that the product identifier correctly identifies the product. In some aspects, the percentage accuracy may comprise 90% or greater. For example, the product-based learning model has been tested to have a greater than 90% prediction and detection accuracy across a several consumer product categories, including oral care, including for over 300 oral care products, product implements, and/or product appliances.

Still further, in some aspects, the product-based learning model (e.g., product-based learning model 107 and/or product-based learning model 107a) can be further trained to filter or distinguish one or more background features (e.g., a bathroom countertop or backsplash) or background products (e.g., non-products or products other than those being imaged or otherwise targeted) from the one or more products and corresponding one or more product identifiers depicted in the pixel data of the plurality of training images. In such aspects, the product-based learning model is trained to detect a given product or portion thereof against background related features (e.g., a cabinet background or one or more non-products or non-interest products) by training the product-based learning model to identify and detect such background features or background products for the removal or distinguishing of such background features or background products in the pixel data.

With further reference to FIG. 3, at block 320, method 300 comprises obtaining, by an imaging app (e.g., imaging app 106 and/or imaging app 106a) executing the one or more processors, a set of one or more images (e.g., images 202a1 and/or 202b1) of the product. The set of one or more images may comprise pixel data as captured by an imaging device (e.g., a mobile device such as computing device 111c1). More generally, the set of one or more images may comprise digital image(s) (e.g., images 202a1 and/or 202b1) as captured by an imaging device (e.g., a digital camera of the mobile device 111c1).

In various aspects, the pixel data as captured by an imaging device (e.g., a mobile device such as computing device 111c1) may depict a product appliance configured to apply the product, or a product implement configured to receive the product. In some example aspects, a product implement may comprise at least one of a manual toothbrush, a battery powered toothbrush, an electrical rechargeable toothbrush, a brush head, a toothbrush refill, a rinsing cup, a tongue scraper, a tongue cleaner, an oral irrigator, a tray, an applicator wand, and/or other oral care products or product components. It is to be understood, however, that additional and/or different product implements, including of additional and/or different product categories (e.g., skin care) are also contemplated herein.

In additional and/or alternative example aspects, a product appliance may comprise at least one of a partial denture, a full denture, a bridge, a veneer, a crown, a cap, orthodontics, an implant, and/or a retainer. It is to be understood, however, that additional and/or different product appliances, including of additional and/or different product categories (e.g., skin care) are also contemplated herein.

With further reference to FIG. 3, at block 330, the method 300 comprises obtaining, by one or more processors, user input including one or more personal parameters associated with the user, one or more goals associated with the user, and (optionally), one or more preferences associated with the user. The personal parameters associated with the user may include one or more of: a current health state associated with the user, one or more dietary factors associated with the user, one or more lifestyle factors associated with the user, one or more demographic factors associated with the user, one or more behavioral or routine factors (e.g., including one or more current oral health habits or routines) associated with the user, or one or more exclusionary factors associated with the user. For instance, the one or more exclusionary factors associated with the user many include one or more of: an allergen, age, gender, pregnancy state, medical recommendation, medical condition, health state, and/or a personal belief. For instance, the current health state associated with the user may be an oral health state, including one or more of: a condition, a sensation, a structural state, a missing component, a tissue trait, an aesthetic state, a dental modification, an oral observation, and/or a sensory state.

The one or more goals associated with the user may include addressing one or more of the following issues: cavities, caries, dental erosion, teeth grinding, bruxism, halitosis, bad breath, tooth staining, tooth yellowing, gingivitis, gum bleeding, gum recession, periodontitis, dry mouth, Xerostomia, plaque, tartar, sensitivity, mouth sores, tooth decay, tooth loss, and/or edentulism.

The one or more preferences associated with the user may include one or more of: a flavor, a texture, a smell, a sensation, a size, a hardness level, a sustainability attribute, an ingredient inclusion, an ingredient exclusion, an oral care product type, an oral care implement type, and/or a packaging type.

Additionally, with further reference to FIG. 3, at block 340, the method 300 comprises predicting, based on the output of a risk factor model (which may be a risk factor learning model, such as the risk factor learning model 108 and/or 108a), one or more risk factors associated with the user. For instance, the imaging app 106 and/or imaging app 106a may access the risk factor model to input the product identifier (from block 320) and the personal parameters associated with the user (from block 330), and the risk factor model may output one or more predicted risk factors associated with the user.

For instance, predicted risk factors associated with the user may include, for example, whether the user is at risk of developing certain oral care conditions in the future, such as gingivitis, periodontitis, tooth decay, yellowing, sensitivity, plaque or tartar build-up, etc. Moreover, the predicted risk factors may include a predicted level of risk and/or a predicted likelihood of the user developing the condition, as well as, in some examples, a predicted amount of time after which the condition is predicted to develop. The predicted risk factors may include consideration of, for example, factors that lead towards maintenance of oral health or improvement of oral health. For example, daily flossing may be a positive factor in determining the risk of plaque build-up.

In some examples, the risk factor model may be an “if/then” model. Preferably, the risk factor model may be a risk factor learning model trained with personal parameters associated with each of a plurality of individuals, and one or more products used by each of the plurality of individuals. That is, the risk factor learning model may be trained to output one or more predicted risk factors associated with each of the plurality of individuals based on the personal parameters associated with each of the plurality of individuals, and the one or more products used by each of the plurality of individuals.

In various aspects, the risk factor learning model can comprise an artificial intelligence (AI) based model trained with at least one AI algorithm. Training of the risk factor learning model involves image analysis of the training data (i.e., personal parameters associated with each of a plurality of individuals, and respective risk factors associated with each of the individuals) to configure weights of the risk factor learning model, and its underlying algorithm (e.g., machine learning or artificial intelligence algorithm) used to predict and/or classify risk factors associated with users based on personal parameters associated with the users. For example, in various embodiments herein, generation of the risk factor learning model involves training the risk factor learning model with the training data, where individuals (and the personal parameters associated with the individuals) are labeled with risk factors associated with each of those individuals.

In some aspects, one or more processors of a server or a cloud-based computing platform (e.g., imaging server 102) may receive the training data via a computer network (e.g., computer network 120). In such embodiments, the server and/or the cloud-based computing platform may train the risk factor learning model with the training data.

In various aspects, as with the product-based learning model discussed above, the risk factor learning model may be trained using a supervised or unsupervised machine learning program or algorithm. The machine learning program or algorithm may employ a neural network, which may be a convolutional neural network, a deep learning neural network, or a combined learning algorithm or program that learns based on features or feature datasets of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on imaging server 102. For example, libraries may include the TENSORFLOW based library, the PYTORCH library, and/or the SCIKIT-LEARN Python library.

As discussed above with respect to the product-based learning model (e.g., product-based learning model 107 and/or product-based learning model 107a), machine learning may involve identifying and recognizing patterns in existing data (in the case of the risk factor learning model, identifying and recognizing patterns in training data, including personal parameters of users associated with particular risk factors, as described herein) in order to facilitate making predictions, classifications, or identification for subsequent data (such as using the risk factor learning model on personal parameters of a new user in order to predict and/or determine and/or identify risk factors associated with the new user).

Furthermore, as discussed above with respect to the product-based learning model (e.g., product-based learning model 107 and/or product-based learning model 107a), machine learning model(s), such as the risk factor learning model as described herein for some embodiments, may be created and trained based upon the training data and one or more additional inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

Moreover, as discussed above with respect to the product-based learning model (e.g., product-based learning model 107 and/or product-based learning model 107a), in unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated. Supervised learning and/or unsupervised machine learning may also comprise retraining, relearning, or otherwise updating models with new, or different, information, which may include information received, ingested, generated, or otherwise used over time. The disclosures herein may use one or both of such supervised or unsupervised machine learning techniques.

In various aspects, the risk factor learning model may be trained, by one or more processors (e.g., one or more processor (e.g., processor 103) of server 102 and/or processors of a computer user device, such as a mobile device) with training data including personal parameters associated with a plurality of users, and respective risk factors associated with each user. In various aspects, the risk factor learning model is configured or otherwise trained to output predictions of risk factors associated with users based on the personal parameters associated with the users. For example, in some aspects, the one or more risk factors are based on prediction(s) of the risk factor learning model, where the model predictions predict, with a degree of predictive accuracy, how likely it is that a particular risk factor is actually associated with a particular user (having particular personal parameters).

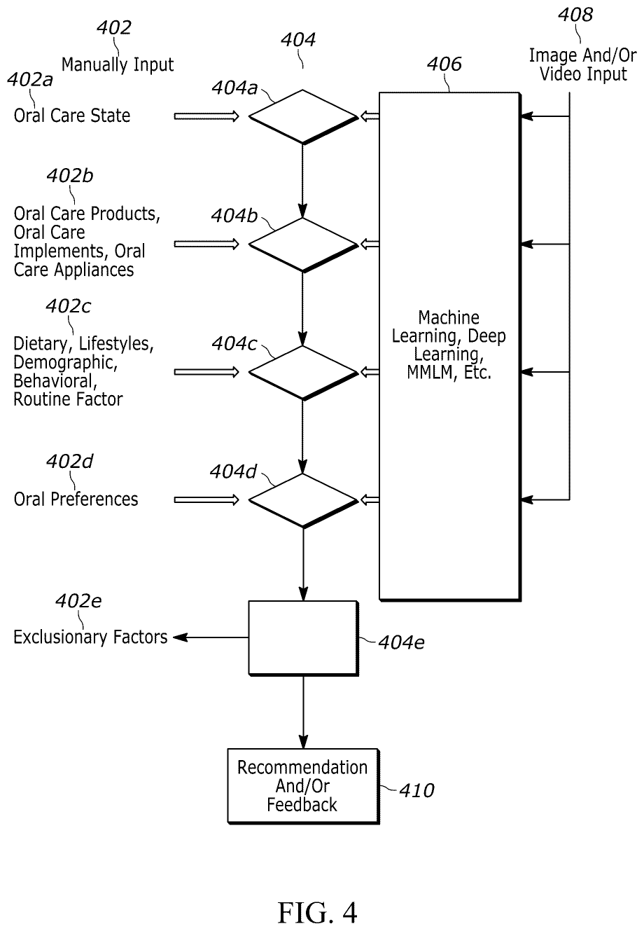

In various aspects, risk factor learning model is accessible by imaging app 106 and/or imaging app 106a, and can be implemented on a computing device (e.g., computing device 111c1) and/or server 102, where computing instructions, implementing on, one or more processors of the computing device (e.g., computing device 111c1) and/or server 102 obtain personal parameters associated with a user (e.g., manually input by the user in some cases, as discussed with respect to FIG. 4).

Additionally, with further reference to FIG. 3, at block 350, the method 300 comprises recommending, based on the output of a recommender model (which may be a recommender learning model, such as the recommender learning model 109 and/or 109a), one or more products and/or one or more routines for the user. For example, the recommended one or more products for the user and/or the recommended one or more routines for the user may include one or more of: an oral care product, an oral care implement, an oral care appliance, an oral routine, a dietary routine, a lifestyle routine, a visit to a dental specialist, a visit to a medical specialist, a modification to an oral care product, a modification to an oral care implement, a modification to an oral routine, a modification to a diet or dietary routine, and/or a modification to a lifestyle or a lifestyle routine. For instance, the imaging app 106 and/or imaging app 106a may access the recommender model to input the product identifier (from block 320), the personal parameters and goals (and optionally, preferences) associated with the user (from block 330), and the predicted risk factors associated with the user (from block 340), and the recommender model may output one or more products and/or one or more routines for the user.

In some examples, the recommender model may be an “if/then” model. Preferably, the recommender model may be a recommender learning model trained with products owned by each of a plurality of users, personal parameters, goals (and optionally, preferences), and predicted risk factors associated with each of the plurality of users, and one or more products and/or routines used by each of the plurality of users. That is, the recommender learning model may be trained to output one or more recommended products and/or routines for each of the plurality of individuals based on the products owned by each of a plurality of users, the personal parameters and goals (and optionally, preferences) associated with each of the plurality of users, and risk factors associated with each of the plurality of users.

In various aspects, the recommender learning model can comprise an artificial intelligence (AI) based model trained with at least one AI algorithm. Training of the recommender learning model involves image analysis of the training data (i.e., products owned by each of a plurality of users, and personal parameters, goals, preferences, and predicted risk factors associated with each of the plurality of users) to configure weights of the risk factor learning model, and its underlying algorithm (e.g., machine learning or artificial intelligence algorithm) used to recommend products and/or routines for users based on the products owned by each of a plurality of users, the personal parameters and goals (and optionally, preferences) associated with each of the plurality of users, and risk factors associated with each of the plurality of users. For example, in various embodiments herein, generation of the recommender learning model involves training the recommender learning model with the training data, where the individuals (and their associated products owned, and personal parameters, goals, preferences, and predicted risk factors) are labeled with recommended products and/or routines associated with each of those individuals.

In some aspects, one or more processors of a server or a cloud-based computing platform (e.g., imaging server 102) may receive the training data via a computer network (e.g., computer network 120). In such embodiments, the server and/or the cloud-based computing platform may train the recommender learning model with the training data.

In various aspects, as with the product-based learning model and risk factor learning model discussed above, the recommender learning model may be trained using a supervised or unsupervised machine learning program or algorithm. The machine learning program or algorithm may employ a neural network, which may be a convolutional neural network, a deep learning neural network, or a combined learning algorithm or program that learns based on features or feature datasets of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on imaging server 102. For example, libraries may include the TENSORFLOW based library, the PYTORCH library, and/or the SCIKIT-LEARN Python library.

As discussed above with respect to the product-based learning model and the risk factor learning model, machine learning may involve identifying and recognizing patterns in existing data (in the case of the recommender learning model, identifying and recognizing patterns in training data, including products owned by each of a plurality of users, the personal parameters, goals, and preferences associated with each of the plurality of users, and risk factors associated with each of the plurality of users, as described herein) in order to facilitate making predictions, classifications, or identification for subsequent data (such as using the recommender learning model on identified products associated with a new user, the personal parameters and goals, and optionally, preferences, associated with the new user, and risk factors associated with the new user).

Furthermore, as discussed above with respect to the product-based learning model and the risk factor learning model, machine learning model(s), such as the recommender learning model as described herein for some embodiments, may be created and trained based upon training data and additional inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

Moreover, as discussed above with respect to the product-based learning model and the risk factor learning model, in unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated. Supervised learning and/or unsupervised machine learning may also comprise retraining, relearning, or otherwise updating models with new, or different, information, which may include information received, ingested, generated, or otherwise used over time. The disclosures herein may use one or both of such supervised or unsupervised machine learning techniques.

In various aspects, the recommender learning model may be trained, by one or more processors (e.g., one or more processor (e.g., processor 103) of server 102 and/or processors of a computer user device, such as a mobile device) with training data including products owned by each of a plurality of users, the personal parameters, goals, and preferences associated with each of the plurality of users, and risk factors associated with each of the plurality of users. In various aspects, the recommender learning model is configured or otherwise trained to output recommended products and/or routines for users based on the products owned by the users, the personal parameters, goals, and preferences associated with each of the users, and risk factors associated with each of the users.

In various aspects, recommender learning model is accessible by imaging app 106 and/or imaging app 106a, and can be implemented on a computing device (e.g., computing device 111c1) and/or server 102, where computing instructions, implementing on, one or more processors of the computing device (e.g., computing device 111c1) and/or server 102 obtain indications of identified products associated with a user (e.g., as determined by the product-based learning model, and/or manually input by the user in some cases, as discussed with respect to FIG. 4), personal parameters, goals, and preferences associated with a user (e.g., manually input by the user in some cases, as discussed with respect to FIG. 4), and risk factors associated with the user (e.g., as determined by the risk factor learning model as discussed above).

With further reference to FIG. 3, at block 360, method 300 further comprises outputting, by the one or more processors, a feedback indication based on the recommended one or more products and/or one or more routines for the user. The feedback indication may comprise, e.g., a visual or audio feedback indication, e.g., as output by a computing device (e.g., computing device 111c1/) depicting, defining, or otherwise indicating the recommended one or more products and/or one or more routines for the user. The feedback indication may further include a rating of the one or more products and/or one or more routines for the user. For instance, to the extent that multiple possible products and/or routines are recommended for the user, the feedback indication may include a rating of the one or more products and/or the one or more routines for the user, which may include one or more of a qualitative rating, a numeric assessment, a visual projection, an augmented reality projection, informational text, and/or a categorical rating associated with the recommended one or more products for the user and/or the recommended one or more routines for the user.

FIG. 4 illustrates an example digital imaging and AI-based method for analyzing images of products, and parameters associated with users provided as user inputs, in order to generate recommendations for users in using the imaging app 106 and/or 106a, product-based learning model 107 and/or 107a, risk factor model 108 and/or 108a, and/or recommender model 109 and/or 109a discussed above, in accordance with various embodiments disclosed herein. As shown with respect to FIG. 4, computing instructions of the imaging app, when executed by one or more processors (e.g., one or more processors 103 of a computing device and/or server 102), may cause the one or more processors to obtain both manual input 402 and image and/or video input 408. The manual input 402 may include various user parameters, such as, for instance, oral care state input 402a, oral care products, oral care implements, and/or oral care appliances input 402b, dietary, lifestyle, demographic, behavioral, routine factor input 402c, oral preferences input 402d, and/or exclusionary factors input 402e. The image and/or video input 408 may include or more images of a product. The one or more processors may analyze the image and/or video input 408 using machine learning, deep learning, MMLM, etc. (e.g., using a product-based learning model, such as the product-based learning model 107 and/or the product-based learning model 107a) at block 406 to identify one or more products.

The identified products may be analyzed in conjunction with the oral care state input 402a at block 404a, analyzed in conjunction with the manually-input oral care products, oral care implements, and/or oral care appliances input 402b at block 404b, analyzed in conjunction with the dietary, lifestyle, demographic, behavioral, routine factor input 402c at block 404c, and/or analyzed in conjunction with the oral preferences input 402d at block 404d. For instance, the analysis at blocks 404a, 404b, 404c, and/or 404d may be performed by one or more of the risk factor model 108a and/or the risk factor model 108, in order to predict one or more risk factors associated with the identified products and the manually entered user parameters. Furthermore, the analysis at blocks 404a, 404b, 404c, and/or 404d may be performed by one or more of the recommender model 109a and/or the recommender model 109, in order to recommend one or more products and/or routines for the user based on the identified products, the predicted risk factors, the one or more risk factors associated with the identified products, and the manually entered user parameters. In some examples, one or more exclusionary factors 402e may be compared against the analyses of blocks 404a, 404b, 404c, and/or 404d in order to generate an analysis taking the exclusionary factors 402e into account at 404e. For instance, exclusionary factors 402e may include allergens, ages, genders, pregnancy states, medical recommendations, medical conditions, health states, and/or personal beliefs, and certain potential recommended products and/or routines may be excluded based on the exclusionary factors. As one example, an alcohol-based product may be excluded from being recommended to a pregnant user or a user with certain personal beliefs. The analyses at blocks 404a, 404b, 404c, 404d, and/or 404e may be used to generate a recommendation and/or feedback including an identification of the product and/or an indication of one or more recommended products and/or routines for the user, at block 410.

FIG. 5 illustrates an example user interface 502 as rendered on a display screen 500 of a user computing device in accordance with various embodiments disclosed herein. For example, as shown in the example of FIG. 5, user interface 502 may be implemented or rendered via an application (app) executing on user computing device 111c1. User interface 502 may be implemented or rendered via a native app executing on user computing device 111c1. In the example of FIG. 5, user computing device 111cl is a user computer device as described for FIG. 1, e.g., where 111c1 is illustrated as an APPLE IPHONE that implements the APPLE IOS operating system and that has display screen 500. User computing device 111c1 may execute one or more native applications (apps) on its operating system, including, for example, imaging app as described herein. Such native apps may be implemented or coded (e.g., as computing instructions) in a computing language (e.g., SWIFT) executable by the user computing device operating system (e.g., APPLE iOS) by the processor of user computing device 111c1.

Additionally, or alternatively, user interface 502 may be implemented or rendered via a web interface, such as via a web browser application, e.g., Safari and/or Google Chrome app(s), or other such web browser or the like.

As shown in the example of FIG. 5, user interface 502 comprises a graphical representation (e.g., of image 202a1 or portion thereof) of a product (e.g., toothpaste). Image 202a1 may comprise the image of the product (or graphical representation thereof) comprising pixel data (e.g., pixel data 202a1p) of at least a portion of the product as described herein.

In various aspects, a feedback indication may be rendered on a display screen (e.g., display screen 500) to indicate (e.g., graphically indicate in the example of FIG. 5), for instance, an identification of the product from the image 202a1 and/or an indication of one or more recommended products (e.g., feedback indication 522) and/or routines (e.g., feedback indication 512) for the user. The feedback indication(s) 512 and/or 522 may include a message 512m to the user designed to provide more further detail regarding the recommended products and/or routines, such as a frequency at which a recommended product is to be applied to address oral care concerns or achieve oral care goals. For instance, as shown in FIG. 5, the message 512m may state: “Based on analysis of the image(s) provided, personal factors, preferences, and goals, an additional whitening product is recommended (shown below), apply three times per week.” Moreover, the feedback indication(s) 512 and/or 522 may include an image of a recommended product 524r (e.g., a recommended product associated with the recommended routine, such as the recommended whitening product mentioned in the message 512m) as determined by the imaging app 106 (e.g., based on the analyses of the product-based learning model 107, risk factor learning model 108, and/or recommender learning model 109) and its related image analysis of image 202a1 and its pixel data and various features.

User interface 502 may further include a selectable UI button 524s to allow the user (e.g., the user of the product shown in image 202a1) to select for purchase or shipment the corresponding product (e.g., manufactured product 524r). In some embodiments, selection of selectable UI button 524s may cause the recommended product(s) to be shipped to the user and/or may notify a third party that the individual is interested in the product(s). For example, either user computing device 111c1 and/or imaging server 102 may initiate, based on the feedback indication(s) 512 and/or 522,1 the manufactured product 524r (e.g., extra whitening toothpaste) for shipment to the user. In such aspects, the product can be packaged and shipped to the user.

In various embodiments, a graphical representation or image (e.g., image 202a1), with the feedback indication(s) 512 and/or 522, may be transmitted, via the computer network (e.g., from an imaging server 102 and/or one or more processors) to user computing device 111c1, for rendering on display screen 500. In other embodiments, no transmission to the imaging server of the user's specific image occurs, where such information or data may instead be generated locally, by the imaging app 106a executing and/or implemented on the user's mobile device (e.g., user computing device 111c1) and rendered, by a processor of the mobile device, on display screen 500 of the mobile device (e.g., user computing device 111c1).

In some embodiments, a recommendation may be rendered on the display screen 500 in real-time or near-real time, during, or after receiving, the set of images and/or input from the user. In embodiments where the image is analyzed by imaging server 102, the image may be transmitted and analyzed in real-time or near real-time by imaging server 102.

In some embodiments, the user may provide a new image that may be transmitted to imaging server 102 for updating, retraining, or reanalyzing by the product-based learning model. In other embodiments, a new image that may be locally received on computing device 111c1 and analyzed, by the product-based learning model, on the computing device 111c1.

In addition, as shown in the example of FIG. 5, the user may select selectable button 512i for reanalyzing (e.g., either locally at computing device 111c1 or remotely at imaging server 102) a new image. Selectable button 512i may cause user interface 502 to prompt the user to attach for analyzing a new image. Imaging server 102 and/or a user computing device such as user computing device 111c1 may receive a new image comprising pixel data of a product. The new image can be captured by the imaging device. The new image (e.g., image 202a1) may comprise pixel data of a product, product dosage, product implement, or other feature(s) as described herein. The product-based learning model, executing on the memory of the computing device (e.g., imaging server 102), may analyze the new image captured by the imaging device to determine a product, product identifier, or other aspects as described herein.

### Additional Considerations

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A digital imaging and artificial intelligence (AI)-based system configured to analyze product images and make product recommendations, the digital imaging and AI-based system comprising:
one or more processors;
an imaging application (app) comprising computing instructions configured to execute on the one or more processors;
a product-based learning model, accessible by the imaging app, and trained with pixel data of a plurality of training images depicting one or more products, the product-based learning model trained to output product predictions of one or more product identifiers corresponding to the one or more products depicted within the pixel data of the plurality of training images;
a risk factor model, preferably wherein the risk factor model is a risk factor learning model, accessible by the imaging app, and trained with personal parameters associated with each of a plurality of individuals, and one or more products used by each of the plurality of individuals, the risk factor model trained to output one or more predicted risk factors associated with each of the plurality of individuals based on the personal parameters associated with each of the plurality of individuals, and the one or more products used by each of the plurality of individuals; and
a recommender model, preferably wherein the recommender model is a recommender learning model, accessible by the imaging app, and trained with the personal parameters associated with each of the plurality of individuals, the one or more products used by each of the plurality of individuals, the one or more predicted risk factors associated with each of the plurality of individuals, one or more goals associated with each of the plurality of individuals, and optionally one or more preferences associated with each of the plurality of individuals, the recommender model trained to output a recommendation of one or more products and/or one or more routines for each of the plurality of individuals;
wherein the computing instructions of the imaging app when executed by the one or more processors, cause the one or more processors to:
obtain a set of one or more images of a product associated with a user, the set of one or more images comprising pixel data as captured by an imaging device, and the pixel data depicting least a portion of the product,
detect, based on output of the product-based learning model inputting the pixel data, a product identifier of the product associated with the user,
obtain user input including one or more personal parameters associated with the user, one or more goals associated with the user, and optionally one or more preferences associated with the user;
predict one or more risk factors associated with the user, based on output of the risk factor model inputting the one or more personal parameters associated with the user and the product identifier of the product;
recommend one or more routines and/or one or more products for the user, based on an output of the recommender model inputting the one or more personal parameters associated with the user, the product identifier of the product associated with the user, the one or more predicted risk factors associated with the user, the one or more goals associated with the user, and optionally the one or more preferences associated with the user; and
output a feedback indication including an indication of the recommended one or more products and/or routines for the user.

2. The digital imaging and AI-based system of claim 1, wherein the one or more product identifiers as output by the product-based learning model are based one or more features identifiable within the pixel data of the plurality of training images, the one or more features comprising: a product category of the one or more products, a product brand of the one or more products, a product variant of the one or more products, a product form of the one or more products, a product packaging of the one or more products, and/or a clinical indication of the product.

3. The digital imaging and AI-based system of claim 2, wherein the product-based learning model is further trained to filter or distinguish one or more background features or background products from the one or more products and corresponding one or more product identifiers depicted in the pixel data of the plurality of training images, and wherein at least a portion of the product is detected, by the product-based learning model, by inputting the pixel data, wherein the pixel data depicts the background features or background products.

4. The digital imaging and AI-based system of claim 1, wherein the product identifier is submitted as an input to look up or link to additional data defining the product as detected by the product-based learning model.

5. The digital imaging and AI-based system of claim 5, wherein the additional data comprises at least one of: formula specification of the product, traits of the products, packaging data of the product, clinical indications of the product.

6. The digital imaging and AI-based system of claim 1, wherein the output of the product-based learning model comprises a product prediction defining a percentage accuracy of 90% or greater that the product identifier correctly identifies the product.

7. The digital imaging and AI-based system of claim 1, wherein detecting the product identifier of the product associated with the user includes detecting one or more identifiers of one or more of implements or appliances.

8. The digital imaging and AI-based system of claim 1, wherein detecting the product identifier of the product associated with the user includes detecting one or more identifiers of one or more implements selected from a manual toothbrush, a battery powered toothbrush, an electrical rechargeable toothbrush, a brush head, a toothbrush refill, a rinsing cup, a tongue scraper, a tongue cleaner, an oral irrigator, a tray, an applicator wand and combinations thereof.

9. The digital imaging and AI-based system of claim 1, wherein detecting the product identifier of the product associated with the user includes detecting one or more identifiers of one or more appliances selected from a partial denture, a full denture, a bridge, a veneer, a crown, a cap, orthodontics, an implant, a retainer and combinations thereof.

10. The digital imaging and AI-based system of claim 1, wherein the personal parameters include one or more of: a current health state associated with the user, one or more dietary factors associated with the user, one or more lifestyle factors associated with the user, one or more demographic factors associated with the user, one or more behavioral or routine factors associated with the user, or one or more exclusionary factors associated with the user.

11. The digital imaging and AI-based system of claim 10, wherein the one or more exclusionary factors associated with the user include an oral health state selected from a condition, a sensation, a structural state, a missing component, a tissue trait, an aesthetic state, a dental modification, an oral observation, a sensory state, and combinations thereof.

12. The digital imaging and AI-based system of claim 1, wherein the one or more preferences associated with the user include one or more of: a flavor, a texture, a smell, a sensation, a size, a hardness level, a sustainability attribute, an ingredient inclusion, an ingredient exclusion, an oral care product type, an oral care implement type, and/or a packaging type.

13. The digital imaging and AI-based system of claim 1, wherein the one or more goals associated with the user include one or more of: cavities, caries, dental erosion, teeth grinding, bruxism, halitosis, bad breath, tooth staining, tooth yellowing, gingivitis, gum bleeding, gum recession, periodontitis, dry mouth, Xerostomia, plaque, tartar, sensitivity, mouth sores, tooth decay, tooth loss, and/or edentulism.

14. The digital imaging and AI-based system of claim 1, wherein the user input includes one or more images or videos associated with the user at two or more time states.

15. The digital imaging and AI-based system of claim 14, wherein the one or more images or videos associated with the user at the two or more time states include images of one or more instances of the user using the product.

16. The digital imaging and AI-based system of claim 1, wherein the feedback indication includes one or more of a qualitative rating, a numeric assessment, a visual projection, an augmented reality projection, informational text, and/or a categorical rating associated with the recommended one or more products for the user and/or the recommended one or more routines for the user.

17. The digital imaging and AI-based system of claim 1, wherein the recommended one or more products for the user and/or the recommended one or more routines for the user include one or more of: an oral care product, an oral care implement, an oral routine, a dietary routine, a 18.
lifestyle routine, a visit to a dental specialist, a visit to a medical specialist, a modification to an oral care product, a modification to an oral care implement, a modification to an oral routine, a modification to a diet or dietary routine, and/or a modification to a lifestyle or a lifestyle routine.
The digital imaging and AI-based system of claim 1, wherein the computing instructions further cause the one or more processors to:
initiate, based on the recommended one or more products for the user, a manufactured product for shipment to a user.
The digital imaging and AI-based system of claim 1, wherein the product-based learning model is an artificial intelligence (AI) based model trained with at least one AI algorithm.
The digital imaging and AI-based system of claim 1, wherein at least one of the one or more processors comprises a processor of a mobile device, and wherein the imaging device comprises a digital camera of the mobile device.
The digital imaging and AI-based system of claim 1, wherein the one or more processors comprises a server processor of a server, wherein the server is communicatively coupled to a computing device via a computer network, and where the imaging app comprises a server app portion configured to execute on the one or more processors of the server and a computing device app portion configured to execute on one or more processors of the computing device, the server app portion configured to communicate with the computing device app portion, wherein the server app portion is configured to implement one or more of: (1) obtaining the set of one or more images of the product; (2) detecting, based on the output of the product-based learning model inputting the pixel data, the product identifier of the product; (3) obtaining the user input; (4) predicting the one or more risk factors associated with the user; (5) recommending the one or more products for the user; (6) recommending the one or more routines for the user; and/or (7) outputting the feedback indication including an indication of the recommended one or more products for the user and the recommended one or more routines for the user.
A digital imaging and artificial intelligence (AI)-based method configured to analyze product images and make product recommendations, the digital imaging and AI-based method comprising:
obtaining, by an imaging application (app) executing on one or more processors, a set of one or more images of a product associated with a user, the set of one or more images comprising pixel data as captured by an imaging device, and the pixel data depicting least a portion of the product; detecting, by the imaging app executing on the one or more processors, based on output of a product-based learning model inputting the pixel data, a product identifier of the product associated with the user, wherein the imaging app accesses the product-based learning model to input the pixel data, and wherein the product-based learning model is trained with pixel data of a plurality of training images depicting one or more products, the product-based learning model trained to output product predictions of one or more product identifiers corresponding to one or more products depicted within pixel data of a plurality of training images;
obtaining, by the imaging app executing on the one or more processors, user input including one or more personal parameters associated with the user, one or more goals associated with the user, and optionally one or more preferences associated with the user;
predicting, by the imaging app executing on the one or more processors, one or more risk factors associated with the user, based on output of a risk factor model inputting the one or more personal parameters associated with the user and the product identifier of the product, wherein the imaging app accesses the risk factor model to input the one or more personal parameters associated with the user and the product identifier of the product, and wherein preferably the risk factor model is a risk factor learning model trained with personal parameters associated with each of a plurality of individuals, and one or more products used by each of the plurality of individuals, the risk factor model trained to output one or more predicted risk factors associated with each of the plurality of individuals based on the personal parameters associated with each of the plurality of individuals, and the one or more products used by each of the plurality of individual;
recommending, by the imaging app executing on the one or more processors, one or more routines and/or one or more products for the user, based on an output of a recommender model inputting the one or more personal parameters associated with the user, the product identifier of the product associated with the user, the one or more predicted risk factors associated with the user, the one or more goals associated with the user, and optionally the one or more preferences associated with the user, wherein the imaging app accesses the recommender model to input the one or more personal parameters associated with the user, the product identifier of the product associated with the user, the one or more predicted risk factors associated with the user, the one or more goals associated with the user, and optionally the one or more preferences associated with the user, and wherein preferably the recommender model is a recommender learning model trained with the personal parameters associated with each of the plurality of individuals, the one or more products used by each of the plurality of individuals, the one or more predicted risk factors associated with each of the plurality of individuals, one or more goals associated with each of the plurality of individuals, and optionally one or more preferences associated with each of the plurality of individuals, the recommender model trained to output a recommendation of one or more products and/or one or more routines for each of the plurality of individuals; and
outputting, by the imaging app executing on the one or more processors, a feedback indication including an indication of the recommended one or more products and/or routines for the user.
A tangible, non-transitory computer-readable medium storing instructions for analyzing product images and making product recommendations, that when executed by one or more processors cause the one or more processors to:
obtain, by an imaging application (app), a set of one or more images of a product associated with a user, the set of one or more images comprising pixel data as captured by an imaging device, and the pixel data depicting least a portion of the product;
detect, by the imaging app, based on output of a product-based learning model inputting the pixel data, a product identifier of the product associated with the user, wherein the imaging app accesses the product-based learning model to input the pixel data, and wherein the product-based learning model is trained with pixel data of a plurality of training images depicting one or more products, the product-based learning model trained to output product predictions of one or more product identifiers corresponding to one or more products depicted within pixel data of a plurality of training images;
obtain, by the imaging app, user input including one or more personal parameters associated with the user, one or more goals associated with the user, and optionally one or more preferences associated with the user;
predict, by the imaging app, one or more risk factors associated with the user, based on output of a risk factor model inputting the one or more personal parameters associated with the user and the product identifier of the product, wherein the imaging app accesses the risk factor model to input the one or more personal parameters associated with the user and the product identifier of the product, and wherein preferably the risk factor model is a risk factor learning model trained with personal parameters associated with each of a plurality of individuals, and one or more products used by each of the plurality of individuals, the risk factor model trained to output one or more predicted risk factors associated with each of the plurality of individuals based on the personal parameters associated with each of the plurality of individuals, and the one or more products used by each of the plurality of individual;
recommend, by the imaging app, one or more routines and/or one or more products for the user, based on an output of a recommender model inputting the one or more personal parameters associated with the user, the product identifier of the product associated with the user, the one or more predicted risk factors associated with the user, the one or more goals associated with the user, and optionally the one or more preferences associated with the user, wherein the imaging app accesses the recommender model to input the one or more personal parameters associated with the user, the product identifier of the product associated with the user, the one or more predicted risk factors associated with the user, the one or more goals associated with the user, and optionally the one or more preferences associated with the user, and wherein preferably the recommender model is a recommender learning model trained with the personal parameters associated with each of the plurality of individuals, the one or more products used by each of the plurality of individuals, the one or more predicted risk factors associated with each of the plurality of individuals, one or more goals associated with each of the plurality of individuals, and optionally one or more preferences associated with each of the plurality of individuals, the recommender model trained to output a recommendation of one or more products and/or one or more routines for each of the plurality of individuals; and
output, by the imaging app, a feedback indication including an indication of the recommended one or more products and/or routines for the user.
